(19) United States
(12) Patent Application Publication
(10) Pub. No.: US 2021/0140971 A1
(43) Pub. Date: May 13, 2021

(54) PREDICTION OF PREECLAMPSIA BASED ON IGFBP-7

(71) Applicant: Roche Diagnostics Operations, Inc., Indianapolis, IN (US)

(72) Inventors: Martin Hund, Rotkreuz (CH); Johann Karl, Penzberg (DE); Ursula-Henrike Wienhues-Thelen, Penzberg (DE)

(21) Appl. No.: 17/152,212

(22) Filed: Jan. 19, 2021

Related U.S. Application Data
(63) Continuation of application No. PCT/EP2019/069581, filed on Jul. 19, 2019.

(30) Foreign Application Priority Data
Jul. 20, 2018 (EP) 18184766.6

(51) Int. Cl. G01N 33/68

(52) U.S. Cl. CPC G01N 33/68; G01N 2496/00; G01N 2800/368; G01N 2800/50

(57) ABSTRACT

The present invention relates to a method for assessing whether a pregnant subject is at risk of developing preeclampsia or a preeclampsia-related condition, or not, said method comprising the steps of determining the amount of the biomarker IGFBP-7 (Insulin-like Growth Factor Binding Protein 7) in a sample from the subject, and comparing the determined amount of the biomarker to a reference. Further, the present invention relates to the in vitro use of the biomarker IGFBP-7, or of at least one detection agent which specifically binds to IGFBP-7 in a sample of a pregnant subject for assessing whether said subject is at risk of developing preeclampsia or a preeclampsia-related condition, or not. Also encompassed by the present invention is a device adapted to carry out the method of the present invention.

### CROSS REFERENCE TO RELATED APPLICATIONS

This application is a continuation application and claims priority to International Patent Application Serial No. PCT/EP2019/069581 (published WO 2020/016441), filed on Jul. 19, 2019, which claims priority to EP Patent Application No. 18184766.6, filed on Jul. 20, 2018, which are both hereby incorporated by reference in their entireties.

### BACKGROUND OF THE DISCLOSURE

The present invention relates to a method for assessing whether a pregnant subject is at risk of developing preeclampsia or a preeclampsia-related condition, or not, said method comprising the steps of determining the amount of the biomarker IGFBP-7 (Insulin-like Growth Factor Binding Protein 7) in a sample from the subject, and comparing the deter-mined amount of the biomarker to a reference. Further, the present invention relates to the in vitro use of the biomarker IGFBP-7, or of at least one detection agent which specifically binds to IGFBP-7 in a sample of a pregnant subject for assessing whether said subject is at risk of developing preeclampsia or a preeclampsia-related condition, or not. Also encompassed by the present invention is a device adapted to carry out the method of the present invention.

Hypertensive disorders represent the most common medical complication of pregnancy, affecting approximately 6 to 8 percent of gestations (Report of the National High Blood Pressure Education Program Working Group on High Blood Pressure in Pregnancy. Am J Obstet Gynecol. 2000; 183(1):S1-S22). Pregnancy complications are, on one hand, associated with pregnancy related mortality of the pregnant woman and, on the other hand, also associated with increased morbidity and mortality of the newborn. In pregnant women above the age of 39 years, maternal mortality at a rate of 14.5 per 100,000 live births is even more frequent.

Hypertensive disorders of pregnancy can be classified as 1) preeclampsia, 2) chronic hypertension (of any cause), 3) chronic hypertension with superimposed preeclampsia, and 4) gestational hypertension (ACOG Task Force on Hypertension in Pregnancy. Obstet Gynecol 2013; 122:1122-31). The clinical management generally includes blood pressure control in the case of preeclampsia and chronic hypertension, seizure prevention in the case of severe hypertension or severe hypertension with eclamptic fit, earlier delivery—34 weeks versus 37 weeks—in the case of chronic hypertension with superimposed preeclampsia and intensive postpartum surveillance in the case of gestational hypertension (NICE (2011) NICE clinical guideline 107: Hypertension in Pregnancy).

Preeclampsia is the most important hypertensive disorder during pregnancy associated with mortality and morbidity for mother and fetus/newborn. (Duley 2009, Semin Perinatol: 33: 130-37).

Preeclampsia is generally defined as pregnancy associated or induced hypertension. It is characterized by hypertension and proteinuria. Details are also found in the standard text books of medicine and the guidelines of the various clinical societies (NICE (2011) NICE clinical guideline 107: Hypertension in Pregnancy).

Currently there are no cures for preeclampsia other than delivery. Preeclampsia can vary in severity from mild to life threatening. A mild form of preeclampsia can be treated with bed rest and frequent monitoring. For moderate to severe cases, hospitalization is recommended and blood pressure medication or anticonvulsant medications to prevent seizures are prescribed. If the condition becomes life-threatening to the mother or the baby, the pregnancy is terminated and the baby is delivered pre-term.

According to current guidelines, diagnosis of preeclampsia is based on the new onset of hypertension and proteinuria after gestational week 20 in pregnant women. A blood pressure greater than or equal to 140 mmHg systolic or greater than or equal to 90 mmHg diastolic on two occasions at least 4 hours apart after 20 weeks of gestation in a woman with a previously normal blood pressure is considered hypertensive. The reliable detection of significant proteinuria is most important in women with new-onset hypertension during pregnancy because it distinguishes between those pregnancies with preeclampsia and those with gestational hypertension and this sets the scene for future monitoring and management. Significant proteinuria is defined internationally as the urinary excretion of more than 300 mg protein in a 24-hour period, and this is included in definitions of preeclampsia (NICE (2011) NICE clinical guideline 107: Hypertension in Pregnancy).

Determination of proteinuria can be realized by 24-hour urine collection, pro-tein/creatinine ratio calculation or dipstick readings. (Executive Summary: Hypertension in Pregnancy, American College of Obstetricians and Gynecologist, Obstet Gynecol 2013; 122:1122-31).

Determination of proteinuria is often done with urine protein dipsticks because the meth-od allows for rapid measurement of proteinuria. However, it is often fraught with incor-rect results and thus a quite inaccurate method to determine kidney dysfunction. Moreo-ver, due to the variability of qualitative determination, this method is discouraged for di-agnostic use and should only be used if other quantitative methods are not available (ACOG Task Force, 2013). Especially amongst women with hypertensive problems, there are high rates of incorrect urinary dipstick results. Of concern, up to 66% of hypertensive women with a negative urinary dipstick were found to have significant proteinuria.” (North R. Classification and diagnosis of preeclampsia. In Preeclampsia: Etiology and Clinical Practice, pages 250-251).

More accurate methods to determine protein in urine are 24 hour urine measurements (generally greater or equal to 300 mg per 24 h urine collection for diagnosis of preeclampsia) or the calculation of protein-creatinine ratio (generally greater than or equal to 0.3, each measured as mg/dl). However, these methods also have certain draw-backs. For example, they are more time-consuming and under certain conditions also error-prone. Bouzari et al. found that proteinuria (determined by 24 h urine measurement) in patients with preeclampsia was associated with adverse outcome in pregnancy. However, it was not an adequate predictor of adverse outcome in preeclampsia (Bouzari Z, Ja-vadiankutenai M, Darzi A, Barat S.: Clin Exp Obstet Gynecol. 2014; 41(2):163-8). Zhang discloses that is not a reliable biomarker for predicting the onset of preeclampsia or HELLP syndrome in pregnant women (Zhang et al. Prediction of adverse outcomes by common definitions of hypertension in pregnancy. Obstet Gynecol 2001; 97:261-7) Similarly, Thangaratinam shows in a systematic review that proteinuria is a poor predictor of complication of preeclampsia (Thangaratinam et al. Estimation of proteinuria as a predictor of complications of pre-eclampsia: a systematic review. BMC Medicine 2009; 7:10).

As preeclampsia is one of the major causes of perinatal morbidity and mortality, there is an urgent need for biomarker for the prediction of the disease. In particular, the prediction of early-onset-preeclampsia is of importance in light of the severe side-effects and the adverse outcomes associated therewith. Moreover, the prediction of preeclampsia is decisive for the planning of preventive or therapeutic intervention studies (Ohkuchi 2011, Hypertension 58: 859-866). On the other hand, patients belonging into a risk group for which an increased risk for preeclampsia within a certain time window can be ruled-out, shall need less special care and, most often, can be treated ambulant (out patient setting).

Doppler ultrasonography has been applied to identify patients with abnormal uterine per-fusion and those patients exhibiting abnormal perfusion identified by Doppler ultrasonography have been suggested to be at risk of developing preeclampsia, eclampsia and/or HELLP syndrome (Stepan 2007, Hypertension, 49: 818-824; Stepan 2008, Am J Obstet Gynecol 198: 175.e1-1). A drawback of Doppler ultrasonography is, however, that highly specialised medical practitioners are required for carrying out and evaluating the results.

Angiogenic factors and antagonists thereof have been suggested to be indicators for preeclampsia. In particular, Placenta growth factor (PlGF), and the soluble fsm-like tyro-sine kinase 1 (sFlt-1) have been reported to be altered in patients suffering from preeclampsia. Individual ratios of sFlt-1 and PlGF at different time points of pregnancy have been individually correlated with a risk for preeclampsia (Kusanovic 2009, J of Maternal—Fetal and Neonatal Medicine 22(11): 1021-1038). WO 2013/068475 discloses a method for diagnosing whether a pregnant subject is at risk for developing preeclampsia within a short period of time based on a first and second ratio of sFlt-1 and PlGF.

IGFBP-7 (Insulin-like growth factor binding protein 7) is a 30-kDa modular glycoprotein known to be secreted by endothelial cells, vascular smooth muscle cells, fibroblasts, and epithelial cells (Ono, Y., et al., Biochem Biophys Res Comm 202 (1994) 1490-1496). It has been described as diagnostic or prognostic markers for various conditions. For example, it has been described as a biomarker for cancer and the use of anti-IGFBP-7 antibodies was suggested as diagnostic tool for detecting neoplastic diseases including tumor angiogenesis (WO 2010/043037). WO 2008/089994 discloses the use of IGFBP-7 in the assessment of heart failure. Urinary IGFBP-7 in combination with TIMP-2 has been shown to be a sensitive and specific biomarker to predict acute kidney injury (AKI) early after cardiac surgery and to predict renal recovery (Meersch et al. PLoS One. 2014 Mar. 27; 9(3)). EP 2 666 872 A1 discloses various markers for the diagnosis and prognosis of renal injury and renal failure. One of the disclosed markers is IGFBP-7.

WO 2017/148854 discloses a method for diagnosing preeclampsia or a preeclampsia-related condition such as eclampsia, HELLP syndrome in a pregnant subject. The docu-ment does not disclose the assessment whether a pregnant subject is at risk of developing preeclampsia or a preeclampsia-related condition. Rather, the document is focused on the identification of subjects who already have developed preeclampsia or a preeclampsia-related condition.

There is a strong need to develop new biomarker based methods to assess the risk of developing preeclampsia or a preeclampsia-related condition, and thus to identify a risk subject as early as possible. In particular, there is a strong need to identify a risk subject be-fore the subject has developed preeclampsia or a preeclampsia-related condition. The early identification would allow the early initiation of suitable patient management measures which aim to address the risk. Thus, a reliable and sensitive biomarker to assess the risk is required.

### BRIEF DESCRIPTION OF THE DISCLOSURE

The technical problem underlying the present invention can be seen as the provision of means and methods for complying with the aforementioned needs. The technical problem is solved by the embodiments characterized in the claims and herein below.

It was found in the context of the studies of the present invention that the measurement of the amount of IGFBP-7 in a sample from a pregnant subject allows for a fast and reliable prediction of preeclampsia or preeclampsia-related conditions (such as eclampsia, or the HELLP syndrome).

Accordingly, the present invention relates to a method for assessing whether a pregnant subject is at risk of developing preeclampsia or a preeclampsia-related condition, or not, said method comprising the steps of

determining the amount of the biomarker IGFBP-7 (Insulin-like Growth Factor Binding Protein 7) in a sample from the subject, and

comparing the determined amount of the biomarker to a reference.

In an embodiment of the present invention, the risk of preeclampsia or a preeclampsia-related condition is assessed by carrying out the further step (c) of assessing whether a pregnant subject is at risk of developing preeclampsia or a preeclampsia-related condition, or not. Said assessment shall be based on the result of the comparison carried out in step (b).

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

The figures show:

FIG. 1: Boxplots for IGFBP-7 levels [ng/mL] in pregnant women divided by control group (no PE=no preeclampsia; n=354) and PE (Preeclampsia; n=27) diagnosis within one week group. The bottom and top edges of each box represent the first and third quartiles, respectively, the band within the box represents the median value, the whiskers represent values that are 1.5 times the interquartile range. The mean value of serum IGFBP-7 is increased in pregnant women with diagnosis of preeclampsia within one week (PE) compared to women without diagnosis of preeclampsia within one week (no PE=control group). Diagnosis of preeclampsia is defined by new onset of hypertension and proteinuria after gestational week 20.

FIG. 2: Boxplots for IGFBP-7 levels [ng/mL] in pregnant women divided by control group (no PE=no preeclampsia; n=328) and PE (Preeclampsia; n=53) diagnosis within four weeks group. The bottom and top edges of each box represent the first and third quartiles, respectively, the band within the box represents the median value, the whiskers represent values that are 1.5 times the interquartile range. The mean value of serum IGFBP-7 is increased in pregnant women with onset of preeclampsia within four weeks (PE) compared to women without onset of preeclampsia within four weeks (no PE=control group).

FIG. 3: Boxplots for IGFBP-7 levels [ng/mL] in pregnant women divided by control group (no PE=no preeclampsia; n=305) and PE (Preeclampsia; n=76) diagnosis during pregnancy until delivery (Overall Diagnosis). The bottom and top edges of each box represent the first and third quartiles, respectively, the band within the box represents the median value, the whiskers represent values that are 1.5 times the interquartile range. The mean value of serum IGFBP-7 is increased in pregnant women with diagnosis of preeclampsia during pregnancy (PE) compared to women without diagnosis of preeclampsia during pregnancy (no PE=control group).

FIG. 4: ROC (Receiver operator characteristic) curve of IGFBP-7 for prediction of preeclampsia within one week. The ROC curve for serum IGFBP-7 for distinguishing pregnant women developing preeclampsia within one week from those not developing preeclampsia within one week resulted in an area under the curve (AUC) of 77.8% (95% confidence interval 69.1-86.5).

FIG. 5: ROC (Receiver operator characteristic) curve of IGFBP-7 for prediction of preeclampsia within four weeks. The ROC curve for serum IGFBP-7 for distinguishing pregnant women developing preeclampsia within four weeks from those not developing preeclampsia within four weeks showed an area under the curve (AUC) of 78.7% (95% confidence interval 72.6-84.8).

### DETAILED DESCRIPTION

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and sub-classification of the subject. The method may be carried out manually or assisted by automation. Preferably, step (a), (b) and/or (c) may in total or in part be assisted by automation, e.g., by a suitable robotic and sensory equipment for the determination in step (a) or a computer-implemented calculation in step (b).

In accordance with the present invention, the risk of preeclampsia or a preeclampsia-related condition in a pregnant subject shall be assessed. Preeclampsia or preeclampsia-related conditions are well-known in the art.

The term “preeclampsia” as used herein refers to a medical condition which is characterized by hypertension and proteinuria. Preeclampsia occurs in pregnant female subjects and the hypertension in that context is also referred to as pregnancy-induced hypertension. Preferably, the pregnancy-induced hypertension is identified to be present in a subject by two blood pressure measurements of 140 mmHg (systolic) and/or 90 mmHg (diastolic) or more, wherein said two measurements have been made at least 6 hours apart. Proteinuria can be identified to be present by 300 mg protein or more in a 24-hour urine sample. Also, proteinuria can be identified by protein dipstick analysis (if ≥2+), if ≥30 mg/dL protein are present in a spot urine sample, or by a protein/creatinine ratio of ≥30 mg protein/mmol creatinine in spot urine.

Preeclampsia in accordance with the present invention can be a mild form or severe form preeclampsia. The terms “mild preeclampsia” and “severe preeclampsia” are well-known in the art. The term “mild preeclampsia” preferably refers to the presence proteinuria and of hypertension (in particular of a blood pressure ≥140/90 mmHg) on 2 occasions, at least 6 hours apart, but without evidence of end-organ damage in a woman who was normotensive before week 20 of gestation. The term “severe preeclampsia” refers to preeclampsia with at least one of the following symptoms, systolic blood pressure of 160 mmHg or higher or diastolic blood pressure of 110 mmHg or higher on 2 occasions at least 6 hours apart, proteinuria of more than 5 g in a 24-hour collection or more than 3+ on 2 random urine samples collected at least 4 hours apart, oliguria (in particular of less than 400 mL urine in 24 hours), persistent headaches, epigastric pain and/or impaired liver function and thrombocytopenia.

The studies carried out in the context of the present invention show that both the risk of early-onset preeclampsia and late-onset preeclampsia can be assessed. In particular, the assessment of the risk of the subject to suffer early-onset-preeclampsia is advantageous as it is, usually, accompanied by more severe side-effects and adverse outcomes compared to the usually relatively mild late-onset-preeclampsia.

In an embodiment, the method of the present invention thus encompasses the assessment of the risk of the subject to suffer from early-onset preeclampsia. Early-onset preeclampsia occurs between about week 20 and about week 34 of gestation. Thus, it is envisaged to obtain the sample between about week 20 and about week 34 of gestation.

In another embodiment, the method of the present invention thus encompasses the assessment of the risk of the subject to suffer from late-onset preeclampsia. Late-onset preeclampsia occurs after week 34 of gestation. Thus, it is envisaged to obtain the sample after week 34 of gestation.

The preeclampsia-related condition is preferably selected from eclampsia and the HELLP syndrome.

Eclampsia is a life-threatening disorder characterized by the appearance of tonic-clonic seizures or coma conditions. Symptoms associated with severe preeclampsia are oliguria of less than 500 ml within 24 hours, cerebral or visual disturbance, pulmonary edema or cyanosis, epigastric- or right upper quadrant-pain, impaired liver function, thrombocytopenia.

The term “HELLP syndrome” is well-known in the art. The HELLP syndrome is a life-threatening obstetric complication usually considered complication of preeclampsia. The HELLP syndrome usually occurs during the later stages of pregnancy. The HELLP syndrome is associated with a high risk of adverse outcomes such as renal failure, subcapsular hepatic hematoma, recurrent preeclampsia, or even death. “HELLP” is an abbreviation of the three main features of the syndrome: Hemolysis, Elevated Liver enzymes, and Low Platelet count. HELLP syndrome can be difficult to diagnose due to the variability of symptoms among patients (frequently patients have no symptoms other than general abdominal pain), and early diagnosis is key in reducing morbidity. If not treated in a timely manner, patients can become critically ill or die due to liver rupture/hemorrhage or cerebral edema. In a patient with possible HELLP syndrome, a batch of blood tests is performed: a full blood count, a coagulation panel, liver enzymes, electrolytes, and renal function studies. Often, fibrin degradation product (FDP) levels are determined, which can be elevated. Lactate dehydrogenase is a marker of hemolysis and is elevated (>600 U/liter).

In accordance with the present invention, it shall be assessed whether a pregnant subject is at risk of developing preeclampsia or a preeclampsia-related condition, or not. Thus, the risk of the pregnant subject to develop preeclampsia or a preeclampsia-related condition (i.e. to suffer from preeclampsia or a preeclampsia-related condition in the future) shall be predicted. Accordingly, the prediction of the risk to develop preeclampsia or a preeclampsia-related condition does not refer to the diagnosis of a currently suffering of the patient from preeclampsia or a preeclampsia-related condition, but refers to the risk of a future development of preeclampsia or a preeclampsia-related condition in the patient. Accordingly, it is envisaged that the subject to be tested does not suffer from the condition to be predicted, in particular at the time point at which the test sample has been obtained.

Preferably, the terms “predicting the risk” or “assessing the risk” as used herein refer to assessing the probability according to which the subject will suffer from preeclampsia or a preeclampsia-related condition. In particular, the risk/probability in a certain time window is predicted, e.g. within three days, one week, two weeks, three weeks, four weeks, or six weeks. Thus, it shall be assessed whether the subject is at short-term risk or not. E.g., the short-term risk is a risk of developing preeclampsia or a preeclampsia-related condition within a period of about one to about four weeks. Also, it is envisaged that the short-term risk is a risk of developing preeclampsia or a preeclampsia-related condition within a period of about one to about two weeks.

In a preferred embodiment of the present invention, the predictive window is a period of one week. Thus, it is assessed whether the subject is at risk of developing preeclampsia or a preeclampsia-related condition, or not, within one week. In a further preferred embodiment of the present invention, the predictive window is a period of two weeks. In a further preferred embodiment of the present invention, the predictive window is a period of three weeks. In another preferred embodiment of the present invention, the predictive window is a period of four weeks. Thus, it is assessed whether the subject is at risk of developing preeclampsia or a preeclampsia-related condition, or not, within four weeks. Preferably, the predictive window is calculated from the time point at which the sample to be tested has been obtained.

As will be understood by those skilled in the art, such a prediction/assessment is usually not intended to be correct for 100% of the subjects. The expression “predicting the risk” typically requires that a prediction/assessment can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort or population.

Also, the expression “predicting the risk” typically requires that the assessment is correct with a negative predictive value as set forth elsewhere herein for a certain portion of subjects (e.g. a cohort in a cohort study). The risk for developing or not developing preeclampsia, or a preeclampsia-related condition in a certain time window in the future can be diagnosed by a test such as the method of the invention with a summary statistic describing the performance of the test with respect to false positive/negative and true positive/negative assessments.

A high negative predictive value indicates a high level of confidence in a negative assessment made by a diagnostic test. The negative predictive value can be expressed as the number of true negative results divided by the sum of the true negative results and the false negative results (i.e. all negative outcomes determined by the diagnostic test). In principle, a negative predictive value can be calculated depending on the sensitivity and specificity of a diagnostic test and the prevalence for a disease or condition in certain cohort. Specifically, the negative predictive value is [(specificity)(1−prevalence)]/[(specificity)(1−prevalence)+(1−sensitivity)(prevalence)]. Prevalence predictions can be obtained from cohort studies whereas case control studies may yield sensitivity and/or specificity for the test. In particular, the negative predictive value of the prediction established by the method of the present invention shall be at least about 80%, at least about 85%, at least about 90%, more preferably, at least about 92% and, most preferably, at least about 94%. The aforementioned negative predictive values e.g. apply to a predictive window of one week.

The positive predictive value (PPV) is the percentage of subjects with a positive test who actually develop preeclampsia or a preeclampsia-related condition. The positive predictive value (PPV) is preferably at least about 20%, more preferably at least about 27%. The aforementioned negative predictive values e.g. apply to a predictive window of four weeks.

In accordance with the present invention, the test subject is allocated either into the group of subjects being at risk of developing preeclampsia or a preeclampsia-related condition, or into the group of subjects being not at risk of developing preeclampsia or a preeclampsia-related condition. A subject who is at risk of developing preeclampsia or a preeclampsia-related condition as referred to in accordance with the present invention, preferably, means that the subject has an elevated risk (within the predictive window). Preferably, said risk is elevated as compared to the average risk in a cohort of pregnant subjects. Accordingly, the phrase “at risk for developing preeclampsia or a preeclampsia-related condition” refers to a pregnant subject which will develop preeclampsia a preeclampsia-related condition within a prognostic time window in the future with a statistically significantly increased likelihood compared to a pregnant subject which is not at risk for developing preeclampsia.

If a subject is not at risk of developing preeclampsia or a preeclampsia-related condition as referred to in accordance with the present invention, preferably, the risk developing preeclampsia or a preeclampsia-related condition is reduced (within the predictive window). Preferably, said risk is reduced as compared to the average risk in a cohort of subjects with.

The “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human subject, i.e. female human pregnant subject. Said subject is preferably after week 19 of gestation. In an embodiment, the pregnant subject is between about week 20 and about week 40 of gestation, in particular, between about week 24 and about week 40 of gestation. In another embodiment, the pregnant subject is between about week 20 and 34 of gestation such as between about week 24 and 34 of gestation. In another embodiment, the pregnant subject is between about week 34 and about week 40 of gestation.

In an embodiment of the present invention, the pregnant subject to be tested suffers from hypertension. Hypertension is defined in this context as blood pressure of 140 mmHg (systolic) and/or 90 mmHg (diastolic) or more at two independent measurements, wherein said two measurements have been made at least 6 hours apart. In an embodiment, the hypertension is new-onset hypertension. Thus, it is envisaged that the pregnant subject shall not have suffered from hypertension before pregnancy.

In an embodiment of the present invention, the subject shows one or more of the following symptoms: new onset of elevated blood pressure, aggravation of pre-existing hypertension, new onset of protein in urine, aggravation of pre-existing proteinuria, epigastric pain, excessive edema/severe swelling, (face, hands, feet), headache, visual disturbances, sudden weight gain (such as more than 1 kg/week in the third trimester), low platelets, elevated liver transaminases, intrauterine growth restriction (or suspected intrauterine growth restriction), abnormal uterine perfusion detected by Doppler sonography with mean pulsatility index >95th percentile in the second trimester and bilateral uterine artery notching.

Further, it is envisaged that the subject is a pregnant subject being older than 40 years and/or a pregnant subject in the first pregnancy, a pregnant subject having a family history of preeclampsia (e.g., preeclampsia in a mother or sister), a pregnant subject having a prior history of preeclampsia in previous pregnancy, a pregnant subject having a body mass index at or above 35 kg/m² at first contact, or a pregnant subject having a multiple pregnancy or pre-existing vascular disease such as hypertension or diabetes, e.g. as described in the NICE (National Institute for Health and Care Excellence, Antenatal Care guideline CG62, March 2008).

In another embodiment of the method of the present invention the subject to be tested is an apparently healthy pregnant subject. In another embodiment, the test subject may be a pregnant subject who does not suffer from proteinuria. In another embodiment, the test subject may be a pregnant subject who does not suffer from hypertension. For such subjects, the method of the present invention can be used in routine screening approaches.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, or any other bodily secretion or derivative thereof. Preferred body fluid samples are urine, blood, serum or plasma. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

Further, it is envisaged that a blood sample is a dried blood spot sample. Dried blood spot samples can be obtained by applying drops of blood onto absorbent filter paper. The blood is allowed to thoroughly saturate the paper and is air dried for several hours. The blood may have been drawn by a lancet from the subject to be tested, e.g. from the finger.

In a preferred embodiment, the sample is a blood (i.e. whole blood), serum or plasma sample. Serum is the liquid fraction of whole blood that is obtained after the blood is allowed to clot. For obtaining the serum, the clot is removed by centrifugation and the supernatant is collected. Plasma is the acellular fluid portion of blood. For obtaining a plasma sample, whole blood is collected in anticoagulant-treated tubes (e.g. citrate-treated or EDTA-treated tubes). Cells are removed from the sample by centrifugation and the supernatant (i.e. the plasma sample) is obtained.

In accordance with the present invention, the amount of Insulin-like Growth Factor Binding Protein 7 (=IGFBP-7) shall be determined. Preferably, the amount of the IGFBP-7 polypeptide is determined. IGFBP-7 is a 30-kDa modular glycoprotein known to be secreted by endothelial cells, vascular smooth muscle cells, fibroblasts, and epithelial cells (Ono, Y., et al., Biochem Biophys Res Comm 202 (1994) 1490-1496). Preferably, the term “IGFBP-7” refers to human IGFBP-7. The sequence of the protein is well-known in the art and is e.g. accessible via Uni-Prot (Q16270, IBP7_HUMAN), or via GenBank (NP_001240764.1). A detailed definition of the biomarker IGFBP-7 is e.g. provided in WO 2008/089994 which herewith is incorporated by reference in its entirety. There are two isoforms of IGFBP-7, Isoform 1 and 2 which are produced by alternative splicing. In an embodiment of the present invention, the total amount of both isoforms is determined (for the sequence, see the UniProt database entry (Q16270-1 and Q16270-2)).

The term “amount” as used herein encompasses the absolute amount of a biomarker as referred to herein, the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts determined from biological read out systems in response to the peptides or intensity signals obtained from specifically bound ligands. It is to be understood that values correlating to the aforementioned amounts or parameters can also be obtained by all standard mathematical operations.

The term “determining” the amount of a biomarker as referred to herein refers to the quantification of the biomarker, e.g. to determining the level of the biomarker in the sample, employing appropriate methods of detection described elsewhere herein.

In an embodiment, the amount of a biomarker is determined by contacting the sample with an agent that specifically binds to the biomarker, thereby forming a complex between the agent and said biomarker, detecting the amount of complex formed, and thereby determining the amount of said biomarker.

The biomarker as referred to herein can be detected using methods generally known in the art. Methods of detection generally encompass methods to quantify the amount of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays, which are commercially available. Further suitable methods to detect biomarker include determining a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

For the detection of biomarker proteins as referred to herein a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker. Sandwich assays are among the most useful immunoassays.

Methods employing electrochemiluminescent labels are well-known. Such methods make use of the ability of special metal complexes to achieve, by means of oxidation, an excited state from which they decay to ground state, emitting electrochemiluminescence. For review see Richter, M. M., Chem. Rev. 104 (2004) 3003-3036.

In an embodiment, the detection antibody (or an antigen-binding fragment thereof) to be used for determining the amount of a biomarker is ruthenylated or iridinylated. Accordingly, the antibody (or an antigen-binding fragment thereof) shall comprise a ruthenium label. In an embodiment, said ruthenium label is a bipyridine-ruthenium(II) complex. Or the antibody (or an antigen-binding fragment thereof) shall comprise an iridium label. In an embodiment, said iridium label is a complex as disclosed in WO 2012/107419.

Determining the amount of a polypeptide (such as IGFBP-7) may, preferably, comprise the steps of (a) contacting the polypeptide with an agent that specifically binds said polypeptide (b) (optionally) removing non-bound agent, (c) determining the amount of bound binding agent, i.e. the complex of the agent formed in step (a). According to a preferred embodiment, said steps of contacting, optionally removing and determining may be performed by an analyzer unit. According to some embodiments, said steps may be performed by a single analyzer unit of said system or by more than one analyzer unit in operable communication with each other. For example, according to a specific embodiment, said system disclosed herein may include a first analyzer unit for performing said steps of contacting and optionally removing and a second analyzer unit, operably connected to said first analyzer unit by a transport unit (for example, a robotic arm), which performs said step of determining.

The agent which specifically binds the biomarker (herein also referred to as “binding agent”) may be coupled covalently or non-covalently to a label allowing detection and measurement of the bound agent. Labeling may be done by direct or indirect methods. Direct labeling involves coupling of the label directly (covalently or non-covalently) to the binding agent. Indirect labeling involves binding (covalently or non-covalently) of a secondary binding agent to the first binding agent. The secondary binding agent should specifically bind to the first binding agent. Said secondary binding agent may be coupled with a suitable label and/or be the target (receptor) of tertiary binding agent binding to the secondary binding agent. Suitable secondary and higher order binding agents may include antibodies, secondary antibodies, and well-known binding-systems such as the streptavidin-biotin system (Vector Laboratories, Inc.). The binding agent or substrate may also be “tagged” with one or more tags as known in the art. Such tags may then be targets for higher order binding agents. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium complexes, iridium complexes, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, avail-able as ready-made stock solution from Roche Diagnostics), CDP-Star™ (Amersham Bio-sciences), ECF™ (Amersham Biosciences). A suitable enzyme-substrate combination may result in a colored reaction product, fluorescence or chemoluminescence, which can be determined according to methods known in the art (e.g. using a light-sensitive film or a suit-able camera system). As for determining the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. A radioactive label can be detected by any method known and appropriate, e.g. a light-sensitive film or a phosphor imager.

The amount of a polypeptide may be, also preferably, determined as follows: (a) contacting a solid support comprising a binding agent for the polypeptide as described elsewhere herein with a sample comprising the peptide or polypeptide and (b) determining the amount of peptide or polypeptide which is bound to the support. Materials for manufacturing supports are well-known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc.

In yet an aspect the sample is removed from the complex formed between the binding agent and one marker prior to the measurement of the amount of formed complex. Accordingly, in an aspect, the binding agent may be immobilized on a solid support. In yet an aspect, the sample can be removed from the formed complex on the solid support by applying a washing solution.

“Sandwich assays” are among the most useful and commonly used assays encompassing a number of variations of the sandwich assay technique. Briefly, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Optionally, any unreacted material may be washed away. The presence of the biomarker is determined by observation of a signal produced by the reporter molecule bound to the detection binding agent. The results may either be qualitative, by simple observation of a visible signal, or may be quantitated by comparison with a control sample containing known amounts of biomarker.

The incubation steps of a typical sandwich assays can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample which is capable of being specifically bound. Further details on how the measurement can be carried out are also found elsewhere herein. The amount of formed complex shall be transformed into an amount of the biomarker reflecting the amount indeed present in the sample.

The terms “binding agent”, “specific binding agent”, “analyte-specific binding agent”, “detection agent” and “agent that specifically binds to a biomarker” are used interchangeably herein. Preferably it relates to an agent that comprises a binding moiety which specifically binds the corresponding biomarker. Examples of “binding agents” or “agents” are a nucleic acid probe, nucleic acid primer, DNA molecule, RNA molecule, aptamer, antibody, antibody fragment, peptide, peptide nucleic acid (PNA) or chemical compound. A preferred agent is an antibody, or antigen-binding fragment thereof, which specifically binds to the biomarker to be determined. The term “antibody” herein is used in the broadest sense and encompasses various antibody structures, including but not limited to monoclonal antibodies, polyclonal antibodies, multispecific antibodies (e.g., bispecific antibodies), and antibody fragments as long as they exhibit the desired antigen-binding activity (i.e. antigen-binding fragments thereof). Preferably, the antibody is a polyclonal antibody. More preferably, the antibody is a monoclonal antibody.

The term “specific binding” or “specifically bind” refers to a binding reaction wherein binding pair molecules exhibit a binding to each other under conditions where they do not significantly bind to other molecules. The term “specific binding” or “specifically binds”, when referring to a protein or peptide as biomarker, refers to a binding reaction wherein a binding agent binds to the corresponding biomarker with an affinity of at least 10⁻⁷ M. The term “specific binding” or “specifically binds” preferably refers to an affinity of at least 10⁻⁸ M or even more preferred of at least 10⁻⁹ M for its target molecule. The term “specific” or “specifically” is used to indicate that other molecules present in the sample do not significantly bind to the binding agent specific for the target molecule.

The term “comparing” as used herein refers to comparing the amount of the biomarker in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer-assisted. Thus, the comparison may be carried out by a computing device. The value of the determined or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired assessment in a suitable output format.

In accordance with the present invention the amount of the biomarker IGFBP-7 shall be compared to a reference. The reference is preferably a reference amount. The term “reference amount” as used herein refers to an amount which allows for allocation of a subject into either (i) the group of subjects suffering from preeclampsia or a preeclampsia-related condition or (ii) the group of subjects not suffering from preeclampsia or a preeclampsia-related condition. A suitable reference amount may be determined from a reference sample to be analyzed together, i.e. simultaneously or subsequently, with the test sample.

Reference amounts can, in principle, be calculated for a cohort of subjects as specified above based on the average or mean values for a given biomarker by applying standard methods of statistics. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity versus specificity pairs resulting from continuously varying the decision threshold over the entire range of data observed. The clinical performance of a prognostic method depends on its accuracy, i.e. its ability to correctly allocate subjects to a certain prognosis. The ROC plot indicates the overlap between the two distributions by plotting the sensitivity versus 1−specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1−specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/1−specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis for a given event with a proper balance of sensitivity and specificity, respectively. Accordingly, the reference to be used for the aforementioned method of the present invention, i.e. a threshold which allows to differentiating between subjects who are at risk of suffering from preeclampsia or a preeclampsia-related condition or those who are not at risk of suffering from preeclampsia or a preeclampsia-related condition among a cohort of pregnant subjects can be generated, preferably, by establishing a ROC for said cohort as described above and deriving a threshold amount therefrom. Dependent on a desired sensitivity and specificity for a diagnostic method, the ROC plot allows deriving a suitable threshold. It will be understood that an optimal sensitivity is desired for excluding a subject who is at risk of suffering from preeclampsia or a preeclampsia-related condition (i.e. a rule out) whereas an optimal specificity is envisaged for a subject to be assessed as being at risk of suffering from preeclampsia or a preeclampsia-related condition (i.e. a rule in).

In certain embodiments, the term “reference amount” herein refers to a predetermined value. Said predetermined value shall allow for differentiating between a subject who is at risk of suffering from preeclampsia or a preeclampsia-related condition and a subject who is at not risk of suffering from preeclampsia or a preeclampsia-related condition.

In an embodiment of the present invention, the reference amount shall allow for ruling-out the risk of suffering from preeclampsia or a preeclampsia-related condition, e.g. within a predictive window of four weeks. An amount of IGFBP-7 which is below this reference amount is typically indicative for a subject who is not at risk of suffering from preeclampsia or a preeclampsia-related condition, e.g. within a predictive window of four weeks.

In an embodiment of the present invention, the reference amount shall allow for ruling in the risk of suffering from preeclampsia or a preeclampsia-related condition, e.g. within a predictive window of one week. An amount of IGFBP-7 which is above this reference amount is typically indicative for a subject who is at risk of suffering from preeclampsia or a preeclampsia-related condition, e.g. within a predictive window of one week.

The following applies as diagnostic algorithm.

Preferably, an amount of IGFBP-7 in the sample of the test subject at or above the reference amount indicates that the subject is at risk to suffer from preeclampsia or a preeclampsia-related condition. Also preferably, an amount of IGFBP-7 in the sample below the reference amount indicates that the subject is not at risk to suffer from preeclampsia and/or a preeclampsia-related condition.

In an embodiment, the reference amount is derived from a pregnant subject or a group of pregnant subjects known not to be at risk to suffer from preeclampsia or a preeclampsia-related condition. Preferably, the reference amount is derived from a pregnant subject or group of pregnant subjects being at the same stage (e.g. trimester, month or week) of gestation as the subject to be tested. Preferably, and amount of the biomarker IGFBP-7 in the sample of the test subject which is decreased as compared to the reference amount or which is identically to the reference amount is indicative for a subject who is not at risk to suffer from preeclampsia or a preeclampsia-related condition.

In an embodiment, the reference amount is derived from a pregnant subject or a group of pregnant subjects known to be at risk to suffer from preeclampsia or a preeclampsia-related condition. Preferably, the reference amount is derived from a pregnant subject or group of pregnant subjects being at the same stage (e.g. trimester, month or week) of gestation as the subject to be tested. Preferably, an amount of the biomarker IGFBP-7 in the sample of the test subject which is increased as compared to the reference amount or which is identically to the reference amount is indicative for a subject who is at risk to suffer from preeclampsia or a preeclampsia-related condition.

The “reference” as used herein typically refers to a reference amount or value which represents a cut-off for making the prediction with a negative predictive value of at least about 80%, at least about 85%, at least about 90%, more preferably, at least about 92% or, most preferably, at least about 94%. In an embodiment, said prediction is made within a window period of one week.

Also, the “reference” as used herein may refer to a reference amount or value which represents a cut-off for making the prediction with a positive predictive value of at least about 20%, more preferably, at least about 27% or, most preferably, at least about 27%. In an embodiment, said prediction is made within a window period of four weeks.

In an embodiment, the reference amount is within a range of about 90 to 110 ng/ml, especially within 90 to 105 ng/ml.

In an embodiment, the reference amount is an amount of about 100 to 105 ng/ml such as an amount of 102 to 105 ng/ml, e.g. 104 ng/ml. Such an amount would e.g. allow for ruling-in the risk. Accordingly, an amount of IGFBP-7 which is above this reference amount is indicative for a subject who is at risk of suffering from preeclampsia or a preeclampsia-related condition, e.g. within a period of one week.

In another embodiment, the reference amount is an amount of about 95 to 99.9 ng/ml such as an amount of 96 to 97 ng/ml, e.g. 97 ng/ml. Such an amount would e.g. allow for ruling-out the risk. Accordingly, an amount of IGFBP-7 which is below this reference amount is indicative for a subject who is not at risk of suffering from preeclampsia or a preeclampsia-related condition, e.g. within a period of four weeks.

Thus, it is also envisaged to use a reference amount which allows for ruling-in the risk and/or a reference amount for ruling-out the risk.

In a further preferred embodiment of the method of the present invention, said method further comprises recommending or initiating a patient management measure based on the assessment made by the method of the present invention

The term “recommending” as used herein means establishing a proposal for a patient management measure or combinations thereof which could be applied to the subject or which must not be applied to the subject. However, in one particular embodiment, it is to be understood that applying the actual management measure, whatsoever, is not comprised by the term. Patient management measures, as used herein, refer to all measures which can be applied to subjects suffering from preeclampsia in order to cure, avoid or handle the health condition. For example, patient management measures include the degree of monitoring (e.g., close, regular or weak monitoring), hospitalization or ambulant maintenance, applying or refraining from drug treatment, or life style recommendations. Preferably, said patient management measure is selected from the group of the following measures if the subject is assessed being at risk for developing preeclampsia or a preeclampsia-related condition: close monitoring, hospitalization, administration of blood pressure reducing agents and life style recommendations. Preferably, said patient management measure is ambulant monitoring if the subject is assessed as being not at risk for developing preeclampsia.

In an embodiment, the blood pressure reducing agent is selected from the group consisting of methyldopa, labetalol and nifedipine.

Also, the present invention contemplates the in vitro use of the biomarker IGFBP-7, or of at least one detection agent which specifically binds to IGFBP-7 in a sample of a pregnant subject for assessing whether said subject is at risk of developing preeclampsia or a preeclampsia-related condition, or not. The term “detection agent” has been defined elsewhere herein. In an embodiment, said detection agent is an antibody, or antigen-binding fragment thereof, which specifically binds to IGFBP-7.

The definitions and explanations given herein above apply mutatis mutandis to the following embodiments of the present invention.

The present invention further relates to a method for differentiating between a pregnant subject who is at risk of developing preeclampsia or a preeclampsia-related condition and a pregnant subject who is not at risk of developing preeclampsia or a preeclampsia-related condition, said method comprising the steps of
determining the amount of the biomarker IGFBP-7 (Insulin-like Growth Factor Binding Protein 7) in a sample from the subject, and
comparing the determined amount of the biomarker to a reference.

The aforementioned method may further comprise step c) of differentiating between a pregnant subject who is at risk of developing preeclampsia or a preeclampsia-related condition and a pregnant subject who is not at risk of developing preeclampsia or a preeclampsia-related condition based on the results of the comparison step b).

Also, the present invention contemplates the in vitro use of the biomarker IGFBP-7, or of at least one detection agent which specifically binds to IGFBP-7 in a sample of a pregnant subject for differentiating between a pregnant subject who is at risk of developing preeclampsia or a preeclampsia-related condition and a pregnant subject who is not at risk of developing preeclampsia or a preeclampsia-related condition.

The present invention further pertains to a computer-implemented method for assessing whether a pregnant subject is at risk of developing preeclampsia or a preeclampsia-related condition, or not, said method comprising
receiving a value for the amount of IGFBP-7 determined in a sample from a pregnant subject at a processing unit,
comparing, by said processing unit, the value received in step (a) to a reference for IGFBP-7, and
assessing whether a pregnant subject is at risk of developing preeclampsia or a preeclampsia-related condition, or not.

The above-mentioned method is a computer-implemented method. Preferably, all steps of the computer-implemented method are performed by one or more processing units of a computer (or computer network). Thus, the assessment in step (c) is carried out by a processing unit. Preferably, said assessment is based on the results of step (b).

The value received in step (a) shall be derived from the determination of the amount of IGFBP-7 in a sample from a pregnant subject as described elsewhere herein. Preferably, the value is a value for the concentration of IGFBP-7. The value will be typically received by the processing unit by uploading or sending the value to the processing unit. Alternatively, the value can be received by the processing unit by inputting the value via an user interface.

In an embodiment of the aforementioned method, the reference set forth in step (b) is established from a memory. Preferably, a value for the reference is established from the memory.

In an embodiment of the aforementioned computer-implemented method of the present invention, the result of the assessment made in step c) is provided via a display, configured for presenting result.

In an embodiment of the aforementioned computer-implemented method of the present invention, the method may comprise the further step of transferring the information on the assessment made in step c) to the individual's electronic medical records.

The present invention further relates to computer program including computer-executable instructions for performing the steps of the computer-implemented method according to the present invention for assessing whether a pregnant subject is at risk of developing preeclampsia or a preeclampsia-related condition, when the program is executed on a computer or computer network. Typically, the computer program specifically may contain computer-executable instructions for performing the steps of the method as disclosed herein. Specifically, the computer program may be stored on a computer-readable data carrier.

The present invention further contemplates a method of aiding in the assessment whether a pregnant subject is at risk of developing preeclampsia or a preeclampsia-related condition, or not, said method comprising the steps of:
receiving a sample obtained from said pregnant subject,
determining the amount of the biomarker IGFBP-7 in said sample, and
providing information on the value for the determined amount of the biomarker IGFBP-7 to a physician, thereby aiding in the assessment whether a pregnant subject is at risk of developing preeclampsia or a preeclampsia-related condition, or not.

The physician shall be the physician who requested the determination of the biomarker IGFBP-7 for the prediction of the risk, i.e. the physician is the attending physician. Said physician shall treat the pregnant subject. The aforementioned method shall aid the attending physician in the assessment whether a pregnant subject is at risk of developing preeclampsia or a preeclampsia-related condition.

In an embodiment of the aforementioned method, step a) of receiving the sample does not encompass the drawing of the sample from the subject. Rather, the sample which has been obtained from the subject (e.g. under supervision of the attending physician) is provided. For example, the sample can be provided by delivering the sample to a laboratory which carries out the determination of the amount of the biomarker IGFBP-7 in said sample.

After the amount has been determined, information on the value for the determined amount is provided to the physician. In addition, information on the value for the reference amount can be provided. The provided information may further contain in indication whether the pregnant subject is at risk or not at risk of developing preeclampsia or a preeclampsia-related condition.

The present invention also relates to a device adapted for assessing whether a pregnant subject is at risk of developing preeclampsia or a preeclampsia-related condition, or not, said device comprising:
an analyzing unit comprising at least one detection agent which specifically binds to the biomarker IGFBP-7, said unit being adapted for determining the amount of said biomarker in a sample of a pregnant subject; and
an evaluation unit comprising a data processor having implemented an algorithm for comparing the amount with a reference, whereby it is assessed whether a pregnant subject is at risk of developing preeclampsia or a preeclampsia-related condition.

The methods of the present invention can be implemented by the aforementioned device. Thus, the device shall be adapted for carrying out the methods of present invention. A device as used herein shall comprise at least the aforementioned units. The units of the device are operatively linked to each other. How to link the units in an operating manner will depend on the type of units included into the device. For example, where means for automatically quantitatively measuring IGFBP-7 are applied in an analyzing unit, the data obtained by said automatically operating unit can be processed by the evaluation unit, e.g., by a computer program which runs on a computer being the data processor in order to facilitate the diagnosis. In an embodiment, the data processor executes the comparison of the amount of the biomarker with the reference.

Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport.

A preferred analyzing unit for determining IGFBP-7 comprises an agent, such as an antibody (or antigen-binding fragment thereof) which specifically recognizes IGFBP-7 as specified elsewhere herein, and a zone for contacting said detection agent with the sample to be tested. The agent may be immobilized on the zone for contacting or may be applied to said zone after the sample has been loaded. The analyzing unit shall be, preferably, adapted for quantitatively determining the amount of complexes of the agent and IGFBP-7.

In a preferred embodiment of the device of the present invention, said stored reference is a predetermined value (as described elsewhere herein). Preferably, an amount of the biomarker IGFBP-7 which is above the reference amount is indicative for a subject who is at risk of suffering from preeclampsia or a preeclampsia-related condition. Preferably, an amount of the biomarker IGFBP-7 which is below the reference amount is indicative for a subject who is not at risk of suffering from preeclampsia or a preeclampsia-related condition.

In another preferred embodiment of the device of the present invention, said stored reference is a reference amount derived from a subject or a group of subjects known to not to be at risk to suffer from preeclampsia or a preeclampsia-related condition, or a reference amount derived from a subject or a group of subjects known to be at risk to suffer from preeclampsia or a preeclampsia-related condition.

All references cited in this specification are herewith incorporated by reference with respect to their entire disclosure content and the disclosure content specifically mentioned in this specification.

### EXAMPLE

The invention will be merely illustrated by the following Examples. The said Examples shall, whatsoever, not be construed in a manner limiting the scope of the invention.

### Example 1

Prediction of Preeclampsia in Pregnant Women within One Week by Determining IGFBP-7 Levels in Maternal Serum or Plasma

An Elecsys IGFBP-7 immunoassay for the fully automated quantification of the analyte IGFBP-7 in serum or plasma on the Cobas® platform (Roche Diagnostics) has been developed.

FIG. 1 displays boxplots for IGFBP-7 levels [ng/mL] in pregnant women divided by control group (no PE=no preeclampsia; n=354) and PE (Preeclampsia; n=27) diagnosis within one week group. The boxplots show the mean value of serum IGFBP-7 is in-creased in pregnant women with diagnosis of preeclampsia within one week (PE; mean IGFBP-7=110.90 ng/mL) compared to women without diagnosis of preeclampsia within one week (no PE=control group; mean IGFBP-7=95.24 ng/mL). Diagnosis of preeclampsia is defined by new onset of hypertension and proteinuria after gestational week 20.

TABLE 1
Maternal serum IGFBP-7 levels [ng/mL] divided by control
group (No PE) and preeclampsia (PE) within one week.

| | | | | | | | | | | | Total |
|---|---|---|---|---|---|---|---|---|---|---|---|
| | Min. | Qu.-05 | Qu.-25 | Median | Qu.-75 | Qu.-95 | Max. | Mean | SD | IQR | N |
| No PE within 1 week | 44.73 | 73.53 | 83.77 | 92.66 | 102.01 | 122.88 | 269.79 | 95.24 | 21.22 | 18.24 | 354 |
| (n = 354) | | | | | | | | | | | |
| PE within 1 week | 77.55 | 89.36 | 99.51 | 107.81 | 120.66 | 143.61 | 174.22 | 110.90 | 19.17 | 21.15 | 27 |
| (n = 27) | | | | | | | | | | | |

FIG. 4 displays the ROC (Receiver operator characteristic) curve of IGFBP-7 for prediction of preeclampsia within one week. The ROC curve for serum IGFBP-7 for distinguishing pregnant women developing preeclampsia within one week from those not developing preeclampsia within one week resulted in an area under the curve (AUC) of 77.8% (95% confidence interval 69.1-86.5).

TABLE 2
Summary table for ROC curve/area under the curve (AUC)
for predicting preeclampsia within one week.

| | AUC | Lower 95% CI | Upper 95% CI |
|---|---|---|---|
| Prediction of PE | 77.8% | 69.1% | 86.5% |
| within one week | | | |

Table 3 shows that the negative predictive value (NPV) for ruling out preeclampsia within one week is 96.6% (95% CI 93.8-98.4) with a sensitivity of 63.0% and a specificity of 80.2% using an IGFBP-7 cutoff of 104.1 ng/mL (maximized sensitivity with a specificity of at least 80%).

TABLE 3
Summary table of predictive performance of maternal
serum IGFBP-7 for predicting preeclampsia within one
week applying an IGFBP-7 cutoff of 104.1 ng/mL.

| | Estimate | 95% CI | Counts |
|---|---|---|---|
| NPV | 96.6% | 93.8; 98.4 | 284/294 |
| PPV | 19.5% | 11.8; 29.4 | 17/87 |
| Sensitivity | 63.0% | 42.4; 80.6 | 17/27 |
| Specificity | 80.2% | 75.7; 84.2 | 284/354 |

### Example 2

Prediction of Preeclampsia in Pregnant Women within Four Weeks by Determining IGFBP-7 Levels in Maternal Serum or Plasma

FIG. 2 displays boxplots for IGFBP-7 levels [ng/mL] in pregnant women divided by control group (no PE=no preeclampsia; n=328) and PE (Preeclampsia; n=53) diagnosis within four weeks group. The boxplots show the mean value of serum IGFBP-7 is in-creased in pregnant women with diagnosis of preeclampsia within one week (PE; mean IGFBP-7=108.75 ng/mL) compared to women without diagnosis of preeclampsia within one week (no PE=control group; mean IGFBP-7=94.34 ng/mL). Diagnosis of preeclampsia is defined by new onset of hypertension and proteinuria after gestational week 20.

TABLE 4
Maternal serum IGFBP-7 levels [ng/mL] divided by control
group (No PE) and preeclampsia (PE) within four weeks.

| | | | | | | | | | | | Total |
|---|---|---|---|---|---|---|---|---|---|---|---|
| | Min. | Qu.-05 | Qu.-25 | Median | Qu.-75 | Qu.-95 | Max. | Mean | SD | IQR | N |
| No PE within 4 weeks | 44.73 | 72.22 | 83.50 | 91.84 | 100.33 | 119.86 | 269.79 | 94.34 | 21.51 | 16.83 | 328 |
| (n = 328) | | | | | | | | | | | |
| PE within 4 weeks | 77.55 | 86.50 | 98.71 | 106.94 | 120.34 | 132.76 | 174.22 | 108.75 | 16.40 | 21.63 | 53 |
| (n = 53) | | | | | | | | | | | |

FIG. 5 displays the ROC (Receiver operator characteristic) curve of IGFBP-7 for prediction of preeclampsia within four weeks. The ROC curve for serum IGFBP-7 for distinguishing pregnant women developing preeclampsia within four weeks from those not developing preeclampsia within four weeks resulted in an area under the curve (AUC) of 78.7% (95% confidence interval 72.6-84.8).

TABLE 5
Summary table for ROC curve/area under the curve (AUC)
for predicting preeclampsia within four weeks.

| | AUC | Lower 95% CI | Upper 95% CI |
|---|---|---|---|
| Prediction of PE | 78.7% | 72.6% | 84.8% |
| within four weeks | | | |

Table 6 shows that the negative predictive value (NPV) for ruling out preeclampsia within four weeks is 94.4% (95% CI 90.7-97.0) with a sensitivity of 75.5% and a specificity of 67.4% using an IGFBP-7 cutoff of 97.6 ng/mL (maximized specificity with a sensitivity of at least 75%).

TABLE 6
Summary table of predictive performance of maternal
serum IGFBP-7 for predicting preeclampsia within four
weeks applying an IGFBP-7 cutoff of 97.6 ng/mL.

| | Estimate | 95% CI | Counts |
|---|---|---|---|
| NPV | 94.4% | 90.7; 97.0 | 221/234 |
| PPV | 27.2% | 20.2; 35.2 | 40/147 |
| Sensitivity | 75.5% | 61.7; 86.2 | 40/53 |
| Specificity | 67.4% | 62.0; 72.4 | 221/328 |

1. A method for assessing whether a pregnant subject is at risk of developing preeclampsia or a preeclampsia-related condition, or not, said method comprising the steps of
(a) determining the amount of the biomarker IGFBP-7 (Insulin-like Growth Factor Binding Protein 7) in a sample from the subject, and
(b) comparing the determined amount of the biomarker to a reference.

2. The method of claim 1, wherein the sample is body fluid selected from the group consisting of blood, serum and plasma.

3. The method of claim 1, wherein the subject is a human subject and/or wherein the subject suffers from hypertension.

4. The method of claim 1, wherein the preeclampsia-related condition is selected from the group consisting of eclampsia and the HELLP syndrome.

5. The method of claim 1, wherein preeclampsia is selected from the group consisting of early-onset preeclampsia and late-onset preeclampsia.

6. The method of claim 1, wherein the risk to be assessed is the short-term risk.

7. The method of claim 6, wherein the short-term risk is a risk of developing preeclampsia or a preeclampsia-related condition within a period of about one to about four weeks.

8. The method of claim 1, wherein the pregnant subject is after about week 19 of gestation.

9. The method of claim 1, further comprising recommending a patient management measure based on the assessment.

10. A computer-implemented method for assessing whether a pregnant subject is at risk of developing preeclampsia or a preeclampsia-related condition, or not, said method comprising
(a) receiving a value for the amount of IGFBP-7 determined in a sample from a pregnant subject at a processing unit,
(b) comparing, by said processing unit, the value received in step (a) to a reference for IGFBP-7, and
(c) assessing whether a pregnant subject is at risk of developing preeclampsia or a preeclampsia-related condition, or not.

11. The method of claim 10, wherein said reference is established from a memory.

12. A method of aiding in the assessment whether a pregnant subject is at risk of developing preeclampsia or a preeclampsia-related condition, or not, said method comprising the steps of:
(a) receiving a sample obtained from said pregnant subject,
(b) determining the amount of the biomarker IGFBP-7 in said sample, and
(c) providing information on the value for the determined amount of the biomarker IGFBP-7 to a physician, thereby aiding in the assessment whether a pregnant subject is at risk of developing preeclampsia or a preeclampsia-related condition, or not.

13. A method for differentiating between a pregnant subject who is at risk of developing preeclampsia or a preeclampsia-related condition and a pregnant subject who is not at risk of developing preeclampsia or a preeclampsia-related condition, said method comprising the steps of
(a) determining the amount of the biomarker IGFBP-7 (Insulin-like Growth Factor Binding Protein 7) in a sample from the subject, and
(b) comparing the determined amount of the biomarker to a reference.

14. (canceled)

15. A device adapted for assessing whether a pregnant subject is at risk of developing preeclampsia or a preeclampsia-related condition, or not, said device comprising:
(a) an analyzing unit comprising at least one detection agent which specifically binds to the biomarker IGFBP-7, said unit being adapted for determining the amount of said biomarker in a sample of a pregnant subject; and
(b) an evaluation unit comprising a data processor having implemented an algorithm for comparing the amount with a reference, whereby it is assessed whether a pregnant subject is at risk of developing preeclampsia or a preeclampsia-related condition.

16. The method of claim 9, wherein the patient management measure (i) is selected from the group of the following measures if the subject has been assessed as being at risk of developing preeclampsia or a preeclampsia-related condition: close monitoring, hospitalization, administration of blood pressure reducing agents and life style recommendations, and (ii) is ambulant monitoring if the subject has been assessed as not being at risk of developing preeclampsia or a preeclampsia-related condition.
